(19) United States
(12) Patent Application Publication
(10) Pub. No.: US 2023/0149586 A1
(43) Pub. Date: May 18, 2023

(54) AIR QUALITY MONITOR WITH INTEGRATED AIR FRESHENER

(71) Applicant: Carrier Corporation, Palm Beach Gardens, FL (US)

(72) Inventors: Michael J. Birnkrant, Wethersfield, CT (US); Callum Bailey, Lakewood Ranch, FL (US); Rajiv Ranjan, Riverview, FL (US); Peter McKinney, Boulder, CO (US)

(21) Appl. No.: 18/055,514

(22) Filed: Nov. 15, 2022

Related U.S. Application Data
(60) Provisional application No. 63/302,239, filed on Jan. 24, 2022.
(60) Provisional application No. 63/264,216, filed on Nov. 17, 2021.

(51) Int. Cl. A61L 9/12

(52) U.S. Cl. CPC A61L 9/122; A61L 2209/111; A61L 2209/14

(57) ABSTRACT

An air monitoring system includes a housing having a housing inlet and a housing outlet. An airflow is configured to pass through the housing inlet into the housing. A sensor is located within the housing. The sensor is configured to monitor at least one of a frequency of dispensation of an air cleaner from an air freshener and a level of a contaminant in the airflow. The air cleaning system is configured to transmit a sensor signal to at least one of the air freshener and a user device to at least one of: recommend a frequency for air freshening, and adjust a frequency of release of the air cleaner from the air freshener.

### CROSS-REFERENCE TO RELATED APPLICATIONS

This application claims the benefit of U.S. Provisional Patent Application No. 63/302,209 filed Jan. 24, 2022 and U.S. Provisional Patent Application No. 63/264,216 filed Nov. 17, 2021, the disclosure of which is incorporated herein by reference in its entirety.

### BACKGROUND

Air fresheners are often sporadic when it comes to the frequency and amount at which air cleaner is released from the air freshener. Air fresheners are often provided as either a stationary time release air freshener or a manual dispensing air freshener. Currently, there is no effective way of indicating that it is time to refreshen the air by dispensing air cleaner from the air freshener. Stationary time release air fresheners may release air cleaner too often or not often enough, based on a simple timer without monitoring the air in which they are dispensing. Manual dispending air fresheners are often dispensed at the whim of the operator, who lacks knowledge (at least beyond personal perceived senses) about the condition of the air they manually dispense the air cleaner in.

### BRIEF DESCRIPTION

In one embodiment, an air monitoring system includes a housing having a housing inlet and a housing outlet. An airflow is configured to pass through the housing inlet into the housing. A sensor is located within the housing. The sensor is configured to monitor at least one of a frequency of dispensation of an air cleaner from an air freshener and a level of a contaminant in the airflow. The air cleaning system is configured to transmit a sensor signal to at least one of the air freshener and a user device to at least one of: recommend a frequency for air freshening, and adjust a frequency of release of the air cleaner from the air freshener.

Additionally or alternatively, in this or other embodiments a fan assembly is located within the housing. The fan assembly is configured to draw an airflow into the housing via the housing inlet.

Additionally or alternatively, in this or other embodiments an air filter is located within the housing, downstream of the inlet.

Additionally or alternatively, in this or other embodiments the air freshener is located within the housing.

Additionally or alternatively, in this or other embodiments the air cleaner includes air cleaner mitigation compounds.

Additionally or alternatively, in this or other embodiments the frequency of release of the air cleaner is automatically adjusted as a result of the sensor signal.

Additionally or alternatively, in this or other embodiments a user-initiated signal is used to adjust a frequency of release of the air cleaner from the air freshener.

Additionally or alternatively, in this or other embodiments the contaminant is one or more of a volatile organic compound (VOC), volatile sulfur compound (VSC) and carbon dioxide (CO₂).

Additionally or alternatively, in this or other embodiments a cleaner outlet is positioned in the housing through which the air cleaner is dispensed.

Additionally or alternatively, in this or other embodiments the air cleaner is one of a gas, mist or vapor.

In another embodiment, a method of monitoring an air condition in a space includes passing an airflow into a housing of an air monitoring system via a housing inlet, sensing one or more contaminants in the airflow via a sensor located in the housing, comparing a level of the one or more contaminants to a threshold, and transmitting a sensor signal to at least one of an air freshener and a user device to at least one of: recommend a frequency for air freshening, and adjust a frequency of release of the air cleaner from the air freshener. The one or more recommendations include a frequency of a dispensation of an air cleaner into the space.

Additionally or alternatively, in this or other embodiments the airflow is urged into the housing via a fan assembly located in the housing.

Additionally or alternatively, in this or other embodiments the airflow is flowed across an air filter located in the housing between the housing inlet and the sensor.

Additionally or alternatively, in this or other embodiments the air cleaner is dispensed from the housing into the room.

Additionally or alternatively, in this or other embodiments the frequency of release of the air cleaner is automatically adjusted as a result of the sensor signal.

Additionally or alternatively, in this or other embodiments the frequency of release of the air cleaner is initiated via a user-initiated signal from a user device.

Additionally or alternatively, in this or other embodiments the one or more contaminants include one or more of a volatile organic compound (VOC), volatile sulfur compound (VSC) and carbon dioxide (CO₂).

Additionally or alternatively, in this or other embodiments the air cleaner comprises air cleaner mitigation compounds.

### BRIEF DESCRIPTION OF THE DRAWINGS

The subject matter, which is regarded as the disclosure, is particularly pointed out and distinctly claimed in the claims at the conclusion of the specification. The following descriptions of the drawings should not be considered limiting in any way. With reference to the accompanying drawings, like elements are numbered alike:

FIG. 1 is a cross-sectional side view of an exemplary air monitoring system including an air freshener disposed within the housing, in accordance with one aspect of the disclosure.

FIG. 2 is a schematic illustration of an exemplary air cleaning system with an air freshener disposed outside of the housing, in accordance with one aspect of the disclosure.

### DETAILED DESCRIPTION

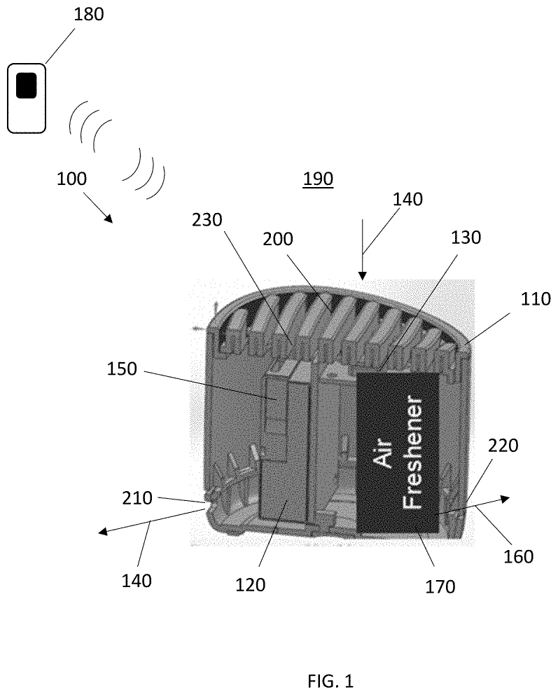

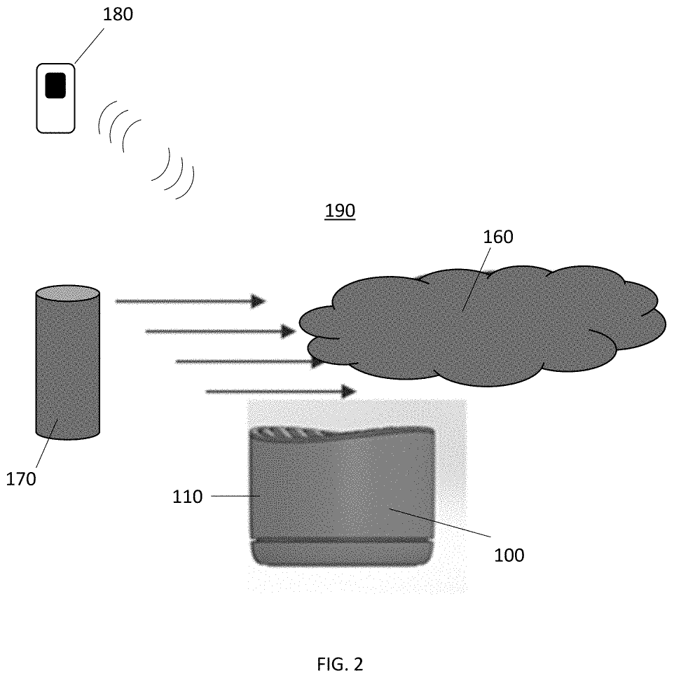

With reference now to the Figures, an exemplary air monitoring system 100 including a housing 110, a fan assembly 130, and a sensor 120 is shown in FIGS. 1 and 2. The air monitoring system 100 is configured to monitor air quality in a space, for example, room 190. FIG. 1 illustrates an embodiment where an air freshener 170 is disposed within the housing 110, while FIG. 2 illustrates an embodiment where the air freshener 170 is disposed outside the housing 110.

Referring again to FIG. 1, the housing 110 includes an inlet 200 and an outlet 210. The fan assembly 130 is disposed within the housing 110. The fan assembly 130 (when present) is configured to draw an airflow 140 into the housing 110 via the inlet 200 and expel the airflow 140 from the housing 110 via the outlet 210. The sensor 120 is disposed within the housing 110 between the inlet 200 and the outlet 210 and is configured to monitor a condition of the airflow 140. In some embodiments, an air filter 230 is disposed downstream of the inlet 200, for example, between the inlet 200 and the sensor 120. In some embodiments, the condition of the airflow 140 may include one or more of a presence of one or more contaminants in the airflow 140, for example, a volatile organic compound (VOC), a volatile sulfur compound (VSC) or a presence of carbon dioxide (CO₂) in the airflow 140. It is to be appreciated that VOC, VSC and CO₂ are exemplary contaminants, and that the sensor may be configured to detect other contaminants, especially odor-causing contaminants.

The sensor 120 is operably connected to a controller 150, which in some embodiments is located in the housing 110. The controller 150 compares a level of contaminants in the airflow 140 to one or more predetermined thresholds. In some embodiments, the predetermined thresholds may differ for each contaminant. For example, a level of VOC in the airflow 140 may be compared to a VOC threshold, and a level of CO₂ in the airflow may be compared to a CO₂ threshold different from the VOC threshold. The controller 150 may categorize levels of contaminants relative to their respective thresholds into one or more contaminant events. The controller 150, in some embodiments, makes one or more recommendations to a user based on one or more of an occurrence, a frequency, or a severity of the contaminant events. The recommendations may include, for example, dispensing of air cleaner 160, in the form of a gas, liquid, mist or vapor, from the air freshener 170, as shown in FIG. 2. The air cleaner 160 may include air cleaner mitigation compounds. In some embodiments, the controller 150 performs the comparison via an algorithm, which may be a machine learning, expert system, or other algorithm.

In some embodiments, the controller 150 communicates the recommendation to a user by a wireless signal to a user device 180, such as a mobile phone, tablet, or other device. The user device 180 may display a detected condition and also one or more recommendations from the controller 150. The user device 180 may prompt the user to act on one or more of the recommendations, such as release of the air cleaner 160 through a cleaner outlet 220 in the housing 110 and into the room 190. Alternatively, the controller 150 may transmit a signal to automatically dispense air cleaner 160 from the air freshener 170, and/or set a time frequency for future dispensing of the air cleaner 160 into the room 190. The controller 150 further monitors the dispensing of the air cleaner 160 via the sensor 120. A time frequency algorithm may be incorporated that utilizes a Fourier transform of the time series data over 1 week, 1 month or 3 months to determine an interval for dispensing or recommendation. The length or time used in the Fourier transform increasing with the period of continuous measurement. In addition, a person can provide an input into the algorithm through interaction with the air monitor or user device to indicate use of an air freshener, called data tags. In a further extension to improve the algorithm, 2 or more data tags identify higher priority Fourier components used in the time frequency algorithm.

In certain instances, the air monitoring system 100 is provided as a way to integrate air fresheners with air monitors (which may have functionality to purify air, and be referred as an ‘air purifier’). For example, sensor data may be used to estimate the mitigation amount/frequency of the air cleaner released from the air freshener. This sensor data may take into consideration air quality classifications (e.g., type of particle, etc.). In certain instances, the classification may include whether the compound being detected by the sensor 120 is a hazardous air pollutant (HAP) (e.g., ammonia, carbon monoxide, polyaromatic hydrocarbons, etc.), a low-risk volatile organic compound (e.g. cooking oil, hand-sanitizer, etc.), or a low-risk mitigation compound (e.g., cyclodextrins, polyamine polymers, perfumes etc.). This classification may enable the ‘self-training’ upon release of the mitigation compounds (i.e., the perfumes) to learn that these mitigation compounds are low-risk VOCs that should not raise alert levels of the sensor 120 (which may be a VOC sensor). This instruction to not raise alerts may be due, at least in part, on the fact that the mitigation compounds are often VOCs themselves, and are not hazardous air pollutants (HAPs). In the case of an aerosolized mitigation delivery (that generate a particulate matter (PM) signal), the onboard particulate monitor can temporarily suppress any alarms caused by particulate droplets originating from the activation of the mitigation aerosol and potentially learn the composition of the aerosol as a low-risk PM fingerprint.

The use of the terms “a” and “and” and “the” and similar referents, in the context of describing the invention, are to be construed to cover both the singular and the plural, unless otherwise indicated herein or cleared contradicted by context. The use of any and all example, or exemplary language (e.g., “such as”, “e.g.”, “for example”, etc.) provided herein is intended merely to better illuminate the invention and does not pose a limitation on the scope of the invention unless otherwise claimed. No language in the specification should be construed as indicating any non-claimed elements as essential to the practice of the invention.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular embodiment disclosed as the best mode contemplated for carrying out this present disclosure, but that the present disclosure will include all embodiments falling within the scope of the claims.

What is claimed is:

1. An air monitoring system comprising:
a housing comprising a housing inlet and a housing outlet, an airflow configured to pass through the housing inlet into the housing; and
a sensor disposed within the housing, the sensor configured to monitor at least one of a frequency of dispensation of an air cleaner from an air freshener and a level of a contaminant in the airflow,
wherein the air cleaning system is configured to transmit a sensor signal to at least one of the air freshener and a user device to at least one of: recommend a frequency for air freshening, and adjust a frequency of release of the air cleaner from the air freshener.

2. The air monitoring system of claim 1, further comprising a fan assembly disposed within the housing, the fan assembly configured to draw an airflow into the housing via the housing inlet.

3. The air monitoring system of claim 1, further comprising an air filter disposed within the housing, downstream of the inlet.

4. The air monitoring system of claim 1, wherein the air freshener is disposed within the housing.

5. The monitoring system of claim 1, wherein the air cleaner comprises air cleaner mitigation compounds.

6. The air monitoring system of claim 1, wherein the frequency of release of the air cleaner is automatically adjusted as a result of the sensor signal.

7. The air monitoring system of claim 1, wherein a user-initiated signal is used to adjust a frequency of release of the air cleaner from the air freshener.

8. The air monitoring system of claim 1, wherein the contaminant is one or more of a volatile organic compound (VOC), volatile sulfur compound (VSC) and carbon dioxide (CO₂).

9. The air monitoring system of claim 1, further comprising a cleaner outlet in the housing through which the air cleaner is dispensed.

10. The air cleaning system of claim 9, wherein the air cleaner is one of a gas, mist, or vapor.

11. A method of monitoring an air condition in a space, comprising:
passing an airflow into a housing of an air monitoring system via a housing inlet;
sensing one or more contaminants in the airflow via a sensor disposed in the housing;
comparing a level of the one or more contaminants to a threshold; and
transmitting a sensor signal to at least one of an air freshener and a user device to at least one of: recommend a frequency for air freshening, and adjust a frequency of release of the air cleaner from the air freshener.

12. The method of claim 11, further comprising urging the airflow into the housing via a fan assembly disposed in the housing.

13. The method of claim 11, further comprising flowing the airflow across an air filter disposed in the housing between the housing inlet and the sensor.

14. The method of claim 11, further comprising dispensing the air cleaner from the housing into the room.

15. The method of claim 11, wherein the frequency of release of the air cleaner is automatically adjusted as a result of the sensor signal.

16. The method of claim 11, wherein the frequency of release of the air cleaner is initiated via a user-initiated signal from a user device.

17. The method of claim 11, wherein the one or more contaminants include one or more of a volatile organic compound (VOC), volatile sulfur compound (VSC) and carbon dioxide (CO₂).

18. The method of claim 11, wherein the air cleaner comprises air cleaner mitigation compounds.
